(19) United States
(12) Patent Application Publication
(10) Pub. No.: US 2005/0250955 A1
(43) Pub. Date: Nov. 10, 2005

(54) Method for producing propenyl oxide using a secondary reactor comprising several feed and/or outlet points

(76) Inventors: Hans-Georg Goebbel, KALLSTADT (DE); Peter Bassler, Viernheim (DE); Joaquim Henrique Teles, Otterstadt (DE); Peter Rudolf, Ladenburg (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/521,782

(22) PCT Filed: Jul. 28, 2003
(86) PCT No.: PCT/EP03/08317

§ 371 Date: Jan. 21, 2005

(30) Foreign Application Priority Data
Jul. 29, 2002 (DE) 102 34 448.5

(51) Int. Cl. C07D 301/19

(52) U.S. Cl. 549/529

(57) ABSTRACT

Process for preparing oxiranes by reacting an organic compound with a hydroperoxide in the presence of a solvent and a catalyst, which comprises at least the steps (i) to (iii): (i) reaction of the hydroperoxide with the organic compound to give a product mixture comprising the reacted organic compound and unreacted hydroperoxide, (ii) separation of the unreacted hydroperoxide from the mixture resulting from step (i), (iii) reaction of the hydroperoxide which has been separated off in step (ii) with the organic compound, wherein the reaction in step (iii) is carried out in a tube reactor which has at least two feed points for the reaction mixture comprising at least the organic compound, the hydroperoxide and the solvent or at least two outlets for the product mixture, or at least two feed points and at least two outlets.

The present invention relates to a process for the catalytic reaction of an organic compound with a hydroperoxide to form an oxirane during the course of which hydroperoxide is separated off and once again reacted with the organic compound in a tube reactor which has at least two feed points and/or two outlets. The novel process allows the running time of the tube reactor configured as a fixed-bed reactor to be advantageously increased in respect of its catalytic activity. In particular, the process of the present invention makes it possible to prepare propylene oxide from propylene and hydrogen peroxide in the presence of methanol as solvent and over a titanium-containing zeolite.

In the customary processes of the prior art, oxiranes are prepared by reaction of suitable organic compounds with hydroperoxides in one or more stages.

For example, the multistage process described in WO 00/07965 carries out the reaction of the organic compound with a hydroperoxide in at least the steps (i) to (iii):
(i) reaction of the hydroperoxide with the organic compound to give a product mixture comprising the reacted organic compound and unreacted hydroperoxide,
(ii) separation of the unreacted hydroperoxide from the mixture resulting from step (i),
(iii) reaction of the hydroperoxide which has been separated off in step (ii) with the organic compound.

Accordingly, the reaction of the organic compound with the hydroperoxide takes place in at least two steps (i) and (iii), with the hydroperoxide which has been separated off in step (ii) being reused in the reaction.

The reactions in steps (i) and (iii) are preferably carried out in two separate reactors, for instance fixed-bed reactors, with the reaction of step (i) preferably taking place in an isothermal reactor and the reaction of step (iii) preferably taking place in an adiabatic reactor. The reactor used in step (i) will hereinafter also be referred to as main reactor and the reactor used in step (ii) will be referred to as after-reactor. According to the process disclosed, the reaction mixture comprising the hydroperoxide and the organic compound are fed into the after-reactor via only one feed point which may, for example, be located at the bottom of the after-reactor. The product mixture is taken from the reactor via only one outlet which is located, for example, at the top of the reactor.

This multistage process can be used generally for the reaction of alkenes as organic compound with hydroperoxides to give oxiranes. In this sequence, the reaction described can also preferably be carried out over a heterogeneous catalyst.

As heterogeneous catalysts, it is possible to use, for example, zeolites, preferably titanium-containing zeolites such as the silicalite TS-1.

However, it is known that the catalytic activity of these catalysts can decrease with running time during the reaction as a result of coating with starting materials or products.

This decrease in the activity can, in particular, become a problem when the reaction is carried out in a fixed-bed reactor. In this case, the catalyst which is bound in the fixed bed cannot be continually replaced or reactivated, since this is always associated with an interruption which interferes in the production process.

To counteract this decrease in the catalytic activity of the catalysts, in particular titanium-containing zeolites such as the zeolite TS-1, it has therefore been proposed that the reaction temperature or the pH of the reaction mixture or both at the same time be adepted to the altered reaction conditions (WO 01/10855).

Methods of reactivating the abovementioned catalysts are also known and are described, for example, in DE-A 197 23 949.8. However, the reaction of the organic compound with the hydroperoxide to form oxirane in the reactor has to be interrupted during the regeneration. Since regeneration of the catalyst in the main reactor is required relatively frequently, installation of at least two of these reactors is also known (WO 01/72729). Cyclic operation of the reactors then enables an advantageous, economical process similar to a continuous process to be achieved.

When the process is carried out using a main reactor and an after-reactor, the catalyst is stressed to a much lesser degree in the after-reactor and is accordingly deactivated to a lesser extent. However, a reactivation as described, for example, in WO 02/22259, which is nevertheless necessary from time to time, is not as simple here as in the case of the main reactor. Owing to the preferred design as an adiabatic reactor, the heat liberated during regeneration cannot be removed, which leads to damage to the catalyst as a result of temperature peaks which occur.

It is an object of the present invention to provide a process by means of which the catalytic activity of the after-reactor can be maintained over very long periods of operation without a deterioration in the propylene oxide selectivity having to be accepted.

To solve precisely this problem in the main reactor, it has already been proposed that the pH of the feed and the reactor temperature be controlled (WO 01/10855). While a change in the pH can also be advantageous for the after-reactor, controlling the reactor temperature proves to be difficult since only the inlet temperature can be controlled in the case of an adiabatically operated reactor. The at least duplicate design of the after-reactor is less economical, since a second reactor would have to be operated only rarely.

We have found that this object is achieved by providing the after-reactor with at least two feed points for the reaction mixture comprising at least the organic compound and hydroperoxide as reactants and the solvent, and/or at least two outlets for the product, with the feed points or outlets being distributed along the reactor and the reactor preferably being operated largely adiabatically.

The present invention accordingly provides a process for preparing oxiranes by reacting an organic compound with a hydroperoxide in the presence of a solvent and a catalyst, which comprises at least the steps (i) to (iii):
(i) reaction of the hydroperoxide with the organic compound to give a product mixture comprising the reacted organic compound and unreacted hydroperoxide,
(ii) separation of the unreacted hydroperoxide from the mixture resulting from step (i),
(iii) reaction of the hydroperoxide which has been separated off in step (ii) with the organic compound,
   wherein the reaction in step (iii) is carried out in a tube reactor which has at least two feed points for the reaction mixture comprising at least the organic compound, the hydroperoxide and the solvent or at least two outlets for the product mixture, or at least two feed points and at least two outlets.

This configuration allows the catalytic activity in the after-reactor to be considerably lengthened compared to the configuration described in the prior art. It is possible to achieve running times which are at least three times as long as in the case of an after-reactor as is used in the prior art. This result could not have been foreseen and is therefore surprising. Thus, complicated regeneration measures for the catalyst are restricted to a minimum in the process of the present invention for preparing oxiranes using an after-reactor having a plurality of feed points and duplicate installation of this after-reactor becomes superfluous. It is sufficient to carry out regeneration measures when the overall plant has been shut down, for example for general maintenance work. The novel process of the present invention is thus extraordinarily useful for industrial production.

For the purposes of the present invention, the term tube reactor refers to a flow tube in which the reaction zone is formed by a cylindrical tube whose length is preferably very much greater than the diameter. The reactants and solvents together enter through at least one of the at least two feed points on the one side of the tube, while a mixture comprising the oxirane as reaction product, unreacted starting materials, the solvent and further components, e.g. by-products formed in the reaction, leaves the tube via one or more outlets on the other side.

The tube reactor can have its longitudinal axis arranged vertically or horizontally. However, its axis is preferably arranged vertically.

Of the at least two feed points, preference is given to at least one being located at one end, namely the upper or lower end of the tube reactor. This is more preferably located at the bottom of the reactor.

Any number of further feed points can be used along the reactor. However, it is generally sufficient to use a maximum of 10 feed points. Preference is given to using a maximum of 5 feed points.

The outlet is preferably located at the top of the reactor. If the reactor is provided with at least two outlets, preference is given to at least one of the outlets being located on the side of the reactor. Further outlets can be distributed along the reactor. Any number of outlets can be used. However, it is generally sufficient to use a maximum of 10 outlets. Preference is given to using a maximum of 5 outlets.

These configurations allow excellent running times of the after-reactor to be achieved in respect of the catalytic activity.

Accordingly, a preferred embodiment of the process of the present invention provides for the tube reactor to have at least one of the features (a) to (f):
(a) its longitudinal axis is arranged vertically,
(b) at least one feed point is located at the bottom of the reactor,
(c) at least one outlet is located at the top of the reactor,
(d) at least one feed point or outlet or feed point and outlet is/are located at the side of the tube reactor,
(e) the number of feed points is not more than 10,
(f) the number of outlets is not more than 10.

In the process of the present invention, the tube reactor is operated as a fixed-bed reactor, i.e. it contains the catalyst composition over which the reactants are reacted. The fixed-bed reactor can be configured as a single-zone reactor. It then generally comprises a single upright tube in which the catalyst composition is arranged without interruption.

However, it is also possible for it to be configured as a multitube reactor. In this arrangement, the single-zone reactor is divided up among a number of thinner tubes.

However, the tube reactor can also be configured as a section reactor. In this configuration, the total catalyst composition is divided among two or more layers.

Owing to the exothermic reaction between the hydroperoxide which has been separated off in step (ii) and the organic compound, the reaction in the after-reactor is preferably carried out under adiabatic conditions, which ensure a readily controllable reaction.

In the process of the present invention, the after-reactor can be supplied with the reaction mixture, hereinafter referred to as feed, comprising at least the organic compound and the hydroperoxide as reactants and the solvent according to different embodiments.

In a preferred embodiment, the total feed is distributed simultaneously over all feed points and introduced into the tube reactor via these.

Accordingly, the reaction mixture is fed into the after-reactor simultaneously via all feed points in this embodiment of the process of the present invention.

However, in a further, preferred embodiment, the feed is firstly fed into the after-reactor exclusively via one feed point. This variant is preferably used when starting up the after-reactor. The feed is preferably fed into the reactor through the uppermost feed point until the conversion is no longer sufficiently high. This can be the case, for example, when the hydroperoxide conversion has dropped below 99.5%. The feed is then switched to the next lower feed point. When the conversion becomes too low at this point as well, the next feed point can be used. If required, the feed can then be switched over the to the next feed point, and so forth.

In this preferred embodiment of the process of the present invention, the reaction mixture is, in particular, fed to the after-reactor via the uppermost feed point and when the hydroperoxide conversion has dropped to a previously defined threshold value, the next lower feed point is used.

In a further embodiment of the process of the present invention, the product mixture is taken off via the bottommost outlet and when the hydroperoxide conversion has dropped to a previously defined threshold value, the next higher outlet is used.

The two above-described embodiments can also be combined. In this preferred embodiment of the process of the present invention, the reaction mixture is fed to the after-reactor via the uppermost feed point and the product mixture is taken off via the bottommost offtake, and when the hydroperoxide conversion has dropped to a previously defined threshold value, the next lower feed point and the next lower outlet are used.

In a further specific embodiment, liquid flows from the bottom upwards through the upright reactor in order to avoid formation of gas cushions. Gas cushions can occur, for example, as a result of the decomposition of hydroperoxides. A known example is the decomposition of hydrogen peroxide to form water and oxygen. Such gas cushions have to be avoided since they present a safety risk. Such gas cushions can also prevent the feed from being distributed uniformly in the fixed bed, which is extremely disadvantageous for constant reaction conditions.

In this embodiment, the abovementioned gas cushions are avoided by part of the feed being introduced as liquid through the bottommost feed point, which is preferably located at the bottom of the reactor. The amount of feed used in this way can be, for example, about 5% by weight of the total feed. However, the liquid employed can also be the solvent used in the process. This is preferably used in an amount of up to about 5% by weight, based on the amount of feed.

In this further, preferred embodiment of the process of the present invention, the main amount of the reaction mixture is fed in via the uppermost feed point and when the hydroperoxide conversion has dropped to a previously defined threshold value, the next lower feed point is used, while at the same time part of the reaction mixture or the solvent is fed in at the bottommost feed point of the reactor.

Each of the feed points is preferably provided with a suitable device by means of which the feed can be distributed uniformly over the cross section of the reactor.

In general, the measures described in the process of the present invention are sufficient to achieve an advantageous increase in the operating life of the catalyst in the after-reactor. However, these measures can of course be supported by the feed being introduced into the reactor at various temperatures or by pH control, as long as this is possible in a simple and economically justifiable fashion. For this purpose, the measures described in WO 01/10855 primarily for the main reactor can also be applied analogously to the after-reactor.

Thus, for example, it is conceivable for at least one acidic or at least one basic compound or a mixture of two or more thereof to be added to the feed to control the pH, with these being able, if appropriate, to be dissolved in a suitable solvent or solvent mixture prior to the addition.

The pH of the feed can also be altered via the starting material streams of which the feed is made up and which flow continuously into the feed during the process. For example, the pH of a solvent stream or a starting material stream comprising the organic compound or else a starting material stream comprising both solvents and organic compound can be altered before addition to the feed and the pH of the reaction mixture in the after-reactor can be influenced in this way.

The pH of the reaction medium in the after-reactor can also be influenced during the reaction via the pH of the hydroperoxide solution which flows into the feed.

As indicated above, modification of the temperature is less well suited to aiding the process of the present invention. However, the temperature of the reaction mixture in the after-reactor can also be influenced via the temperature of the feed stream.

To support the process of the present invention, it is in principle possible to vary the temperature and the pH of the feed separately during the reaction of the hydroperoxide with the organic compound in the after-reactor. However, it is also possible for the pH and temperature to be altered simultaneously.

If pH and temperature changes are to be employed for supporting the process of the present invention, a constant activity of the heterogeneous catalyst is generally achieved by the temperature of the reaction medium in the after-reactor being increased with increasing running time. Whether the pH of the reaction medium has to be increased or decreased, depends essentially on the type of catalysts used. If, as in a preferred embodiment of the process of the present invention, a titanium-containing silicalite, for example the silicalite TS-1, is used as heterogeneous catalyst, the pH generally has to be reduced during the course of the reaction to keep the activity of the catalyst constant.

It is of course conceivable for, for example, one or more temperature increases or one or more pH reductions to be carried out during the course of the reaction in order to bring the activity and selectivity of the catalyst to a prescribed standard value.

In general, the temperature of the feed at the feed point is in the range from 0 to 120° C., preferably in the range from 10 to 100° C. and more preferably in the range from 20 to 90° C. The pH is generally in the range from 2 to 6 and particularly preferably in the range from 3 to 6. In long-term operation, the temperature can also be altered by preferably 2° C./day or less, more preferably by from 0.2 to 1.0° C./day and the pH can be altered by preferably 0.5 units/day or less, more preferably by from 0.01 to 0.2 units/day, in order to support the process of the present invention. The pressures occurring in the after-reactor range from 1 to 100 bar, preferably from 1 to 40 bar, more preferably from 1 to 30 bar.

Apart from the parameters, temperature and pH of the reaction medium, the pressure under which the reaction takes place can also be varied for the purposes of the present invention. However, preference is given to employing pressures under which no gas phase is present.

In an embodiment of the process of the present invention, therefore, the reaction mixture fed into the tube reactor has a pH of from 2 to 6 and a temperature of from 0 to 120° C. and the pressure in the tube reactor is from 1 to 100 bar.

The concentration of the organic compound to be reacted is generally selected so that the molar ratio of organic compound to be reacted to hydroperoxide in the feed is preferably in the range from 0.7 to 20, more preferably in the range from 0.8 to 5.0, particularly preferably in the range from 0.9 to 2.0 and in particular in the range from 1.0 to 1.6.

In this case, the reaction is carried out at a pressure, a temperature and a residence time of the reaction mixture so that hydroperoxide conversions of generally above 90%, preferably above 92% and particularly preferably in the range from 95 to 99.5%, are achieved.

The residence times of the reaction mixture in the after-reactor depend essentially on the desired conversions. In general, they are less than 5 hours, preferably less than 3 hours, more preferably less than 1 hour and particularly preferably about half an hour.

The process of the present invention for preparing oxiranes can be carried out using the starting materials known from the prior art.

Preference is given to using organic compounds which have at least one C-C double bond. Examples of such organic compounds having at least one C-C double bond include the following alkenes:

ethene, propene, 1-butene, 2-butene, isobutene, butadiene, pentenes, piperylene, hexenes, hexadienes, heptenes, octenes, diisobutene, trimethylpentene, nonenes, dodecene, tridecene, tetradecene to eicosene, tripropene and tetrapropene, polybutadienes, polyisobutenes, isoprene, terpenes, geraniol, linalool, linalyl acetate, methylenecyclopropane, cyclopentene, cyclohexene, norbomene, cycloheptene, vinylcyclohexane, vinyloxirane, vinylcyclohexene, styrene, cyclooctene, cyclooctadiene, vinylnorbomene, indene, tetrahydroindene, methylstyrene, dicyclopentadiene, divinylbenzene, cyclododecene, cyclododecatriene, stilbene, diphenylbutadiene, vitamin A, beta carotene, vinylidene fluoride, allyl halides, crotyl chloride, methallyl chloride, dichlorobutene, allyl alcohol, methyl alcohol, butenols, butenediols, cyclopentenediols, pentenols, octadienols, tridecenols, unsaturated steroids, ethoxyethene, isoeugenols, anethole, unsaturated carboxylic acids such as acrylic acid, methacrylic acid, crotonic acid, maleic acid, vinylacetic acid, and also unsaturated fatty acids such as oleic acid, linoleic acid, palmitic acid, also naturally occurring fats and oils.

Preference is given to using alkenes containing from 2 to 8 carbon atoms. Particularly preferably, ethene, propene and butane are reacted. Especially preferably, propene is reacted.

As hydroperoxide, it is possible to use the known hydroperoxides which are suitable for the reaction with the organic compound. Examples of such hydroperoxides are tert-butyl hydroperoxide and ethylbenzene hydroperoxide. Preference is given to using hydrogen peroxide as hydroperoxide for the oxirane synthesis, with an aqueous hydrogen peroxide solution also being able to be used.

The preparation of hydrogen peroxide can be carried out using, for example, the anthraquinone process. This process is based on the catalytic hydrogenation of an anthraquinone compound to form the corresponding anthrahydroquinone compound, subsequent reaction of this with oxygen to form hydrogen peroxide and subsequent extraction to separate off the hydrogen peroxide formed. The catalysis cycle is closed by renewed hydrogenation of the anthraquinone compound which is obtained back.

An overview of the anthraquinone process is given in “Ullmann's Encyclopedia of Industrial Chemistry”, 5th edition, volume 13, pages 447 to 456.

It is likewise conceivable to obtain hydrogen peroxide by converting sulfuric acid into peroxodisulfuric acid by anodic oxidation with simultaneous evolution of hydrogen at the cathode. Hydrolysis of the peroxodisulfiric acid then leads via peroxomonosulfuric acid to hydrogen peroxide and sulfuric acid, which is thus recovered.

It is of course also possible to prepare hydrogen peroxide from the elements.

The synthesis of the oxirane by reaction of the hydroperoxide with the organic compound is carried out in the presence of a solvent and one or more suitable catalysts. Preference is in turn given to heterogeneous catalysts.

All heterogeneous catalysts which are suitable for the respective reaction are conceivable. Preference is given to using catalysts which comprise a porous oxidic material, e.g. a zeolite. Preference is given to using catalysts which comprise a titanium-, germanium-, tellurium-, vanadium-, chromium-, niobium- or zirconium-containing zeolite as porous oxidic material.

Specific mention may be made of titanium-, germanium-, tellurium-, vanadium-, chromium-, niobium- and zirconium-containing zeolites having a pentasil zeolite structure, in particular the types which can be assigned X-ray-crystallographically to the ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, CLO, CON, CZP, DAC, DDR, DFO, DFT, DOH, DON, EAB, EDI, EMT, EPI, ERI, ESV, EUO, FAU, FER, GIS, GME, GOO, HEU, IFR, ISV, ITE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structure or to mixed structures comprising two or more of the abovementioned structures. Furthermore, titanium-containing zeolites having the ITQ-4, SSZ-24, TTM-1, UTD-1, CIT-1 or CIT-5 structure are also conceivable for use in the process of the present invention. Further titanium-containing zeolites which may be mentioned are those of the ZSM-48 or ZSM-12 structure.

Particular preference is given to using Ti zeolites having an MFI or MEL structure or an MFI/MEL mixed structure. Very particular preference is given to the titanium-containing zeolite catalysts which are generally referred to as “TS-1”, “TS-2”, “TS-3” and also Ti zeolites having a framework structure isomorphous with β-zeolite.

The use of a heterogeneous catalyst comprising the titanium-containing silicalite TS-1 is very advantageous.

It is possible to the use the porous oxidic material itself as catalyst. However, it is of course also possible for the catalyst used to be a shaped body comprising the porous oxidic material. All processes known from the prior art can be used for producing the shaped body from the porous oxidic material.

Noble metals in the form of suitable noble metal components, for example in the form of water-soluble salts, can be applied to the catalyst material before, during or after one or more shaping steps in these processes. This method is preferably employed for producing oxidation catalysts based on titanium silicates or vanadium silicates having a zeolite structure, and it is thus possible to obtain catalysts which contain from 0.01 to 30% by weight of one or more noble metals from the group consisting of ruthenium, rhodium, palladium, osmium, iridium, plantinum, rhenium, gold and silver. Such catalysts are described, for example, in DE-A 196 23 609.6.

The shaped bodies can also be processed further. All methods of comminution are conceivable, for example splitting or crushing the shaped bodies, as are further chemical treatments as are described above by way of example.

If one or more shaped bodies is/are used as catalyst, it/they can, after deactivation has occurred, be regenerated by a method in which the deposits responsible for deactivation are burned off in a targeted manner. This is preferably carried out in an inert gas atmosphere containing precisely defined amounts of oxygen-donating substances. This method can be applied particularly advantageously to catalysts of the zeolite type. The regeneration process mentioned is described in DE-A 197 23 949.8. It is also possible to use the regeneration processes mentioned there in the discussion of the prior art.

As solvents, preference is given to using all solvents which completely or at least partly dissolve the starting materials used in the oxirane synthesis. For example, it is possible to use water; alcohols, preferably lower alcohols, more preferably alcohols having less than 6 carbon atoms, for example methanol, ethanol, propanols, butanols, pentanols, diols or polyols, preferably those having less than 6 carbon atoms; ethers such as diethyl ether, tetrahydrofuran, dioxane, 1,2-diethoxyethane, 2-methoxyethanol; esters such as methyl acetate or butyrolactone; amides such as dimethylformamide, dimethylacetamide, N-methyl-pyrrolidone; ketones such as acetone; nitriles such as acetonitrile; sulfoxides such as dimethyl sulfoxide; aliphatic, cycloaliphatic and aromatic hydrocarbons, or mixtures of two or more of the abovementioned compounds.

Preference is given to using alcohols. The use of methanol as solvent is particularly preferred.

In the process of the present invention, it is also possible to react a plurality of organic compounds with the hydroperoxide. It is likewise conceivable for a plurality of hydroperoxides to be used for the reaction. If a plurality of organic compounds and/or a plurality of hydroperoxides are reacted with one another in the respective steps, various products resulting from the reactions may be present in the mixtures. The oxiranes formed can, if desired, be separated by distillation.

In an embodiment of the process of the present invention, the pH or/and the temperature of the reaction mixture in the after-reactor can also be altered.

If the pH of the reaction mixture is to be set by altering the pH of the hydroperoxide solution which is added to the feed, the stream comprising the hydroperoxide which has been separated off in step (ii) can, for example, be treated in an appropriate way to alter the pH.

The pH of a hydroperoxide stream, in particular the hydrogen peroxide solution, can be adjusted by customary methods. Care just has to be taken to ensure that the addition of acidic or basic compounds or addition of a solution comprising acidic or basic compounds to the hydroperoxide solution does not have an adverse effect on the subsequent reaction with the organic compound with hydroperoxide. In particular, the pH of the hydroperoxide solution can be altered by treatment of the hydroperoxide solution with at least one ion exchanger, by addition of an acidic, basic or neutral compound or a mixture of two or more thereof to the hydroperoxide solution or by a combination of these methods.

Both strongly basic and weakly basic compounds or both strongly acidic and weakly acidic compounds are in principle suitable here. Particular mention may be made of the following salts:

ammonium salts, alkali metal salts, especially lithium, sodium and potassium salts, and also alkaline earth metal salts. The anions of these salts include, for example, halides such as chloride and bromide, nitrate, sulfate or hydroxide and also the anions of phosphorus-, arsenic-, antimony- and tin-containing acids, e.g. perchlorate, phosphate, hydrogenphosphate, dihydrogenphosphate, arsenate and stannate. Other anions such as formate, acetate, hydrogencarbonate or carbonate are also conceivable.

Examples which may be mentioned include lithium chloride, lithium bromide, sodium bromide, lithium nitrate, sodium nitrate, potassium nitrate, lithium sulfate, sodium sulfate, potassium sulfate, sodium hydroxide, potassium hydroxide, ammonium hydroxide, sodium carbonate, sodium hydrogencarbonate, potassium carbonate, lithium carbonate, potassium hydrogencarbonate, lithium hydrogencarbonate and potassium hydrogenphosphate and also lithium, magnesium, calcium, barium or ammonium acetate. Mention may likewise be made of carboxylates of carboxylic acids, in particular carboxylic acids having from 1 to 10 carbon atoms, and also alkoxides of alcohols having from 1 to 10 carbon atoms. Further examples include ammonium dihydrogenphosphate, sodium dihydrogenphosphate, potassium dihydrogenphosphate, disodium dihydrogenpyrophosphate.

As ion exchangers, it is in principle possible to use all ion exchangers known to those skilled in the art, for example organic ion exchangers, for instance those based on polystyrene, or inorganic ion exchangers, for instance hydrotalcites and other sheet silicates which may contain exchangeable carbonate, hydrogencarbonate or hydroxide groups.

Examples of basic ion exchangers which are particularly preferred for the purposes of the present invention are polystyrene resins bearing tertiary amine groups, for instance the commercially available anion exchangers Lewatit® MP62 and Lewatit® MP63 and also Dowex® MWA/1 and Dowex® AMW-500. Furthermore, the use of polystyrene resins containing quaternary ammonium groups and having hydroxide counterions is also conceivable. Examples which may be mentioned are the commercially available exchangers Lewatit® OC-1950 and also Dowex® 1, Dowex® 2, Dowex® 11, Dowex® 21K and Dowex® 550A.

However, it is also possible to add the abovementioned compounds to the feed, either directly or as a solution in a solvent before the feed is fed into the after-reactor.

The temperature of the feed can be controlled, for example, by the temperature of at least one starting material stream which is introduced into the feed.

As main reactor for the oxirane synthesis in the process of the present invention, it is of course possible to use all conceivable reactors which are best suited to the respective reactions. For the purposes of the oxirane synthesis, a reactor is not restricted to a single vessel. Rather, it is also possible to use, for example, a cascade of stirred vessels. Preference is given to using at least two reactors connected in parallel, as described in WO 01/72729.

The main reactor used for the oxirane synthesis is preferably a fixed-bed reactor. Further preference is given to using a fixed-bed tube reactor as fixed-bed reactor.

In particular, an isothermal fixed-bed reactor is used as reactor for step (i) in the process of the present invention for preparing oxiranes and an adiabatic fixed-bed tube reactor is used in step (iii).

The hydroperoxide is preferably separated off in step (ii) by distillation in a column.

Preference is given to not only the hydroperoxide but also the oxirane being separated off directly from the reaction mixture in the same separation apparatus. In this intermediate isolation by distillation, the oxirane is then separated off from the mixture via the top of the column. The hydroperoxide is separated off via the side offtake of the column. However, it is also possible, in a further embodiment of the process, to separate off the hydroperoxide from the mixture not via the side offtake of the column but instead via the outlet at the bottom of the column. It usually further comprises water and the solvent which is used for the reaction.

The oxirane distilled off at the top of the column is contaminated with low boilers which are volatile under the distillation conditions, for example unreacted organic compound, water or solvent. It then usually has to be subjected to a purification step, for instance a further distillation in a column which is connected in series with the column used as separation apparatus, in order to be obtained in the purity necessary for further use.

The product mixture obtained from the after-reactor in step (iii) also usually has to be subjected to a distillation step to isolate the oxirane. For this purpose, it can be transferred to a distillation apparatus in a manner analogous to the above-described procedure. However, it is also possible to combine it with the product mixture obtained from step (ii) and to obtain the oxirane therefrom by distillation.

In a particularly preferred embodiment of the process of the present invention, propylene is used as organic compound and hydrogen peroxide is used as hydroperoxide and the reaction is carried out in methanol as solvent over a heterogeneous catalyst comprising a titanium-containing zeolite. Thus, a particularly preferred embodiment of the process of the present invention comprises the preparation of propylene oxide using an after-reactor having a plurality of feed points.

Here, the hydrogen peroxide conversion in step (i) reaches about 85% to 90% and that in step (iii) reaches about 95% based on step (ii). An overall hydrogen peroxide conversion of about 99% over the two steps can be achieved at a propylene oxide selectivity of about 94-95%.

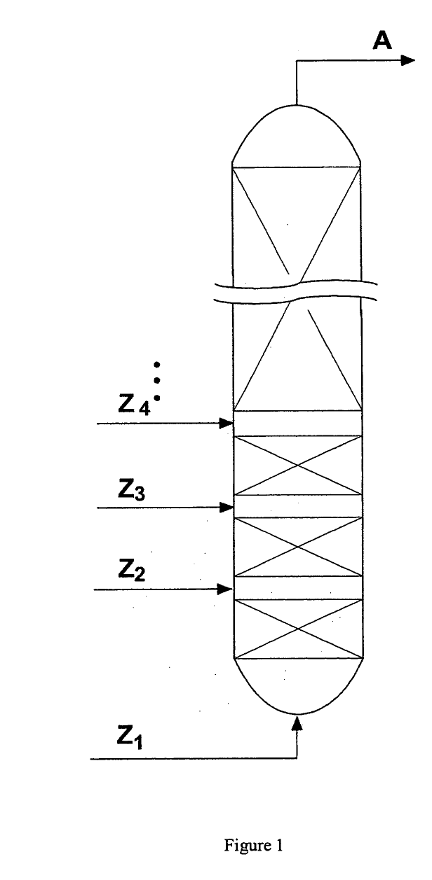

The invention is illustrated by way of example with the aid of FIGS. 1 to 3.

FIG. 1 shows a reactor having a feed point Z₁ at the bottom, an outlet A at the top and further feed points Z₂, Z₃, Z₄, . . . at the side.

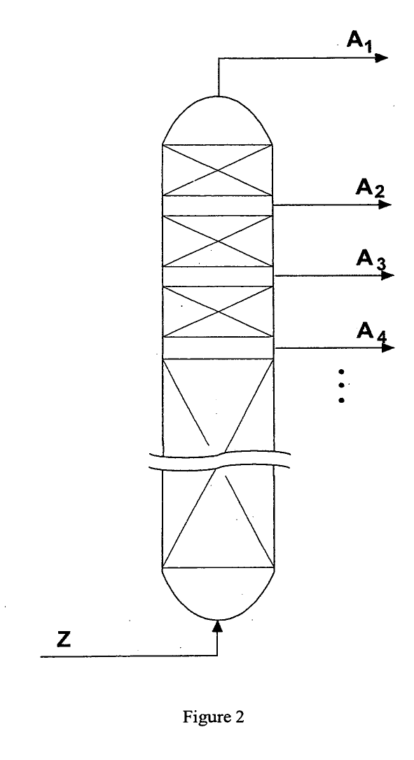

However, an arrangement as outlined in FIG. 2 is also possible. Here, the feed point Z is located at the bottom of the reactor, an outlet A₁ is located at the top and further outlets A₂, A₃, A₄, . . . are located at the side of the reactor.

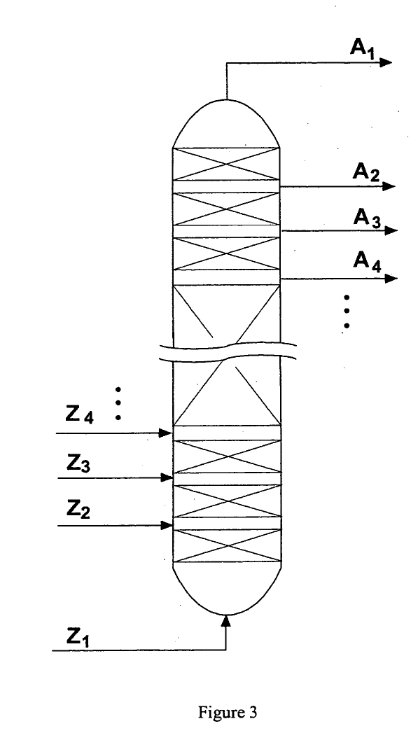

FIG. 3 shows a reactor having a feed point Z₁ at the bottom, an outlet A₁ at the top and further feed points Z₂, Z₃, Z₄, . . . and outlets A₂, A₃, A₄, . . . at the side.

The feed comprising the reactants is introduced into the reactor via one or more of the feed points Z, Z₁, Z₂, Z₃, Z₄, . . . , and the product mixture leaves it via one or more of the outlets A, A₁, A₂, A₃, A₄, . . . Appropriate choice of inlets and outlets allows, as described above, reaction zones to be selected in the respective reactor so that the catalytic activity always remains largely constant.

Horizontal and diagonal or indicated diagonal lines in the reactors symbolize fixed beds or packings or layers comprising catalyst composition.

1-11. (canceled)

12. A process for preparing an oxirane by reacting an organic compound with a hydroperoxide in the presence of a solvent and a catalyst, which comprises:
(i) reacting the hydroperoxide with the organic compound to give a product mixture comprising a reacted organic compound and an unreacted hydroperoxide,
(ii) separating the unreacted hydroperoxide from the product mixture resulting from step (i), and
(iii) reacting the unreacted hydroperoxide which has been separated off in step (ii) with the organic compound,
wherein the reaction in the steps (i) and (iii) is carried out in two separate reactors and the reaction in step (iii) is carried out in an adiabatic tube reactor which has at least two feed points for a reaction mixture comprising at least the organic compound, the hydroperoxide and the solvent, or at least two outlets for the product mixture, or at least two feed points and at least two outlets, wherein at least one feed point is located at the bottom of the reactor, at least one outlet is located at the top of the reactor, and at least one feed point or outlet or feed point and outlet is/are located at the side of the reactor.

13. The process as claimed in claim 12, wherein an isothermal fixed-bed reactor is used in step (i) and a fixed-bed reactor is used as tube reactor in step (iii).

14. The process as claimed in claim 12, wherein the tube reactor has at least one of the features:
(a) the longitudinal axis of the tube reactor is arranged vertically,
(e) the number of feed points is not more than 10, and
(f) the number of outlets is not more than 10.

15. The process as claimed in claim 12, wherein the reaction mixture is fed into the tube reactor simultaneously via all feed points.

16. The process as claimed in claim 12, wherein the reaction mixture is fed into the tube reactor exclusively via the uppermost feed point and after the hydroperoxide conversion has dropped to a predefined threshold value, the reaction mixture is fed in via the next lower feed point.

17. The process as claimed in claim 12, wherein the product mixture is taken from the tube reactor exclusively via the bottommost outlet and when the hydroperoxide conversion has dropped to a previously defined threshold value, the product mixture is taken off via the next higher outlet.

18. The process as claimed in claim 12, wherein part of the reaction mixture or of the solvent is fed in simultaneously with the reaction mixture at the bottommost feed point of the tube reactor.

19. The process as claimed in claim 12, wherein each feed point is provided with a device by means of which the reaction mixture is uniformly distributed over the entire cross section of the tube reactor.

20. The process as claimed in claim 12, wherein the reaction mixture fed into the tube reactor has a pH of from 2 to 6 and a temperature of from 0 to 120° C. and the pressure in the tube reactor is from 1 to 100 bar.

21. The process as claimed in claim 12, wherein propylene is used as the organic compound, hydrogen peroxide is used as the hydroperoxide, the oxirane is propylene oxide and the reaction is carried out in methanol as the solvent over a heterogeneous catalyst comprising a zeolite.

22. The process as claimed in claim 21, wherein the zeolite is TS-1.

23. The process as claimed in claim 21, wherein an isothermal fixed-bed reactor is used in step (i) and a fixed-bed reactor is used as tube reactor in step (iii),
wherein the tube reactor has at least one of the features:
(a) the longitudinal axis of the tube reactor is arranged vertically,
(e) the number of feed points is not more than 10, and
(f) the number of outlets is not more than 10,
and wherein the reaction mixture fed into the tube reactor has a pH of from 2 to 6 and a temperature of from 0 to 120° C. and the pressure in the tube reactor is from 1 to 100 bar.

24. The process as claimed in claim 21, wherein the reaction mixture is fed into the tube reactor simultaneously via all feed points.

25. The process as claimed in claim 21, wherein the reaction mixture is fed into the tube reactor exclusively via the uppermost feed point and after the hydroperoxide conversion has dropped to a predefined threshold value, the reaction mixture is fed in via the next lower feed point.

26. The process as claimed in claim 21, wherein the product mixture is taken from the tube reactor exclusively via the bottommost outlet and when the hydroperoxide conversion has dropped to a previously defined threshold value, the product mixture is taken off via the next higher outlet.

27. The process as claimed in claim 21, wherein part of the reaction mixture or of the solvent is fed in simultaneously with the reaction mixture at the bottommost feed point of the tube reactor.

28. The process as claimed in claim 21, wherein each feed point is provided with a device by means of which the reaction mixture is uniformly distributed over the entire cross section of the tube reactor.

29. A process for preparing an oxirane by reacting an organic compound with a hydroperoxide in the presence of a solvent and a catalyst, which comprises:
(i) reacting the hydroperoxide with the organic compound to give a product mixture comprising a reacted organic compound and an unreacted hydroperoxide,
(ii) separating the unreacted hydroperoxide from the product mixture resulting from step (i),
(iii) reacting the unreacted hydroperoxide which has been separated off in step (ii) with the organic compound,
wherein the reaction in the steps (i) and (iii) is carried out in two separate reactors and the reaction in step (iii) is carried out in an adiabatic tube reactor which has at least two feed points for a reaction mixture comprising at least the organic compound, the hydroperoxide and the solvent, or at least two outlets for the product mixture, or at least two feed points and at least two outlets, wherein at least one feed point is located at the bottom of the reactor, at least one outlet is located at the top of the reactor, and at least one feed point or outlet or feed point and outlet is/are located at the side of the reactor,
and wherein propylene is used as the organic compound, hydrogen peroxide is used as the hydroperoxide, the oxirane is propylene oxide and the reaction is carried out in methanol as the solvent over a heterogeneous catalyst comprising a zeolite, and wherein the zeolite is TS-1,
and wherein the reaction mixture fed into the tube reactor has a pH of from 2 to 6 and a temperature of from 0 to 120° C. and the pressure in the tube reactor is from 1 to 100 bar.

30. The process as claimed in claim 29, wherein an isothermal fixed-bed reactor is used in step (i) and a fixed-bed reactor is used as tube reactor in step (iii).

31. The process as claimed in claim 29, wherein the tube reactor has at least one of the features:
(a) the longitudinal axis of the tube reactor is arranged vertically,
(e) the number of feed points is not more than 10, and
(f) the number of outlets is not more than 10.
